(19) United States
(12) Patent Application Publication
(10) Pub. No.: US 2013/0230835 A1
(43) Pub. Date: Sep. 5, 2013

(54) Method for Simplifying the Donning of Refastenable Disposable Absorbent Articles

(71) Applicants: Kimberly Louise Ellefson, Oshkosh, WI (US); Charles Lewis Cooke, JR., Neenah, WI (US); Kristina Fries Smits, Menasha, WI (US); Shannon Kathleen Melius, Appleton, WI (US); Suzanne Marie Schmoker, Oshkosh, WI (US); Eric Brian Maclean, Appleton, WI (US)

(72) Inventors: Kimberly Louise Ellefson, Oshkosh, WI (US); Charles Lewis Cooke, JR., Neenah, WI (US); Kristina Fries Smits, Menasha, WI (US); Shannon Kathleen Melius, Appleton, WI (US); Suzanne Marie Schmoker, Oshkosh, WI (US); Eric Brian Maclean, Appleton, WI (US)

(21) Appl. No.: 13/411,960

(22) Filed: Mar. 5, 2012

(51) Int. Cl. A61F 13/49; G09B 23/28; A61F 13/84

(52) U.S. Cl. 434/262; 604/386; 604/385.11; 206/494

(57) ABSTRACT

A method for simplified donning of a refastenable garment. The method includes configuring the article such that the first transverse side is open and such that the second transverse side is closed; thereafter positioning and fastening the first front and back side panels around a first leg; thereafter opening the second transverse side; thereafter positioning and fastening the second front and back side panels around a second leg; and thereafter drawing the article up into a wear position on the subject. Also disclosed is a method for simplifying the application of a refastenable garment that includes providing specific instructions to a user, such as via graphical images. Also disclosed is a package of refastenable garments, the package including secondary information for simplifying the application of a refastenable garment to a wearer.

### BACKGROUND

Many absorbent articles intended for personal wear, such as diapers, training pants, feminine hygiene products, adult incontinence products, bandages, medical garments and the like, are designed to be sufficiently absorbent to pull moisture from liquid body exudates including urine, menses, blood, etc. away from the wearer to reduce skin irritation caused by prolonged wetness exposure.

Training pants and adult incontinence products can come pre-assembled in a wear configuration to more closely resemble conventional underpants. In particular, front and back waist regions of such training pants are typically attached at a seam either permanently or refastenably (such as by a primary fastening system) to define a wear configuration of the pants having a waist opening and leg openings.

For such articles where the attachment is refastenable, such as diapers, training pants, and incontinence underwear, a caregiver can find such articles difficult to apply to the subject without removing the subject's shoes and bottom garments. This is particularly applicable to active children of toilet training age, and of adults of limited mobility. Similarly, an individual caring for himself or herself can find such articles difficult to apply to himself or herself without removing his or her shoes and bottom garments. Although attempts have been made to develop techniques by which a garment can be applied to a user without the need to remove shoes and bottom garments, such techniques have been inadequate.

### SUMMARY

In one aspect, the present invention relates to a method for applying a refastenable absorbent article to a subject. In one embodiment of the method, the article has first and second transverse sides, the first transverse side having first front and back side panels and the second transverse side having second front and back side panels. The method includes configuring the article such that the first transverse side is open and such that the second transverse side is closed; thereafter positioning the first front and back side panels around a first leg such that the first front side panel meets the first back side panel, and thereafter fastening the first front side panel to the first back side panel; thereafter opening the second transverse side; thereafter positioning the second front and back side panels around a second leg such that the second front side panel meets the second back side panel, and thereafter fastening the second front side panel to the second back side panel; and thereafter drawing the article up into a wear position on the subject.

In particular embodiments, the positioning around the first and second legs includes positioning around the first and second thighs, respectively. In particular embodiments, the positioning around the first and second thighs occurs while the subject is sitting. In particular embodiments, the configuring includes beginning with a configuration in which both the first and second transverse sides are closed, and thereafter opening the first transverse side. In particular embodiments, the article comprises a first waist region and second waist region, and the configuring comprises separating the first waist region from the second waist region by tearing along one or more lines of perforations.

In another aspect, the invention relates to a method for simplifying the application of a refastenable absorbent article to a subject. In one embodiment of the method, the article has first and second transverse sides, the first transverse side having first front and back side panels and the second transverse side having second front and back side panels. The method includes manufacturing a plurality of refastenable absorbent articles, and assembling the plurality of articles into a package. The method further includes instructing a consumer to configure the article such that the first transverse side is open and such that the second transverse side is closed; to position the first front and back side panels around a first leg such that the first front side panel meets the first back side panel, and to thereafter fasten the first front side panel to the first back side panel; to thereafter open the second transverse side; and to thereafter position the second front and back side panels around a second leg such that the second front side panel meets the second back side panel, and to thereafter fasten the second front side panel to the second back side panel.

In particular embodiments, the plurality of refastenable absorbent articles are assembled into the package in a closed configuration. In other embodiments, the plurality of refastenable absorbent articles are assembled into the package in an open configuration. In particular embodiments, the instructing includes providing graphic images, such as graphic images disposed on the package. Particular embodiments further comprise instructing the consumer to tear along a line of weakness so as to configure the article such that the first transverse side is open and such that the second transverse side is closed.

In another embodiment, the method includes providing a first graphic image associated with the package illustrating positioning the first front and back side panels around the first leg such that the first front side panel meets the first back side panel, wherein the second transverse side is depicted as closed; providing a second graphic image associated with the package illustrating opening the second transverse side; and providing a third graphic image associated with the package illustrating positioning the second front and back side panels around the second leg such that the second front side panel meets the second back side panel. The first graphic image is shown sequentially before the second graphic image, and the second graphic image is shown sequentially before the third graphic image. Particular embodiments of the method further include providing a fourth graphic image associated with the package illustrating drawing the article up into a wear position on the subject, and at least one of the graphic images includes a depiction of the subject without bottom garments removed.

In yet another aspect, the invention relates to a package including secondary information for simplifying the application of a refastenable absorbent article to a subject. In particular embodiments, the article has first and second transverse sides, the first transverse side having first front and back side panels and the second transverse side having second front and back side panels. The package includes an outer surface; a plurality of refastenable absorbent articles disposed within the package; a first graphic image associated with the package illustrating positioning the first front and back side panels around a first leg such that the first front side panel meets the first back side panel, wherein the second transverse side is depicted as closed; a second graphic image associated with the package illustrating opening the second transverse side; and a third graphic image associated with the package illustrating positioning the second front and back side panels around a second leg such that the second front side panel meets the second back side panel. The first graphic image is shown sequentially before the second graphic image, and the second graphic image is shown sequentially before the third graphic image. In particular embodiments, each refastenable absorbent article of the plurality of refastenable absorbent articles is disposed within the package in a closed configuration.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 depicts a front perspective view of one example of a garment suitable for use in conjunction with particular embodiments of the present invention, the garment shown in a prefastened, pant-like configuration.

FIG. 2 depicts a plan view of the garment of FIG. 1, the garment shown in an unfastened, laid-open, relaxed configuration.

FIG. 3 depicts a side perspective view of another example of a garment suitable for use in conjunction with particular embodiments of the present invention, the garment shown in a partially prefastened, pant-like configuration.

FIG. 4 depicts a top view of one embodiment of a section of a manufacturing method suitable for making garments akin to the garment of FIG. 1.

FIG. 5 depicts a top view of another embodiment of a section of a manufacturing method suitable for making certain garments suitable for use in conjunction with particular embodiments of the present invention.

FIG. 6 depicts a top view of one embodiment of a section of a manufacturing method suitable for making garments akin to the garment of FIG. 3.

FIG. 7 is a perspective view of a package of refastenable absorbent garments, the package including exemplary instructional graphic images.

FIG. 8A is a perspective view of a garment similar to that shown in FIG. 1, depicted with both refastenable side seams closed.

FIG. 8B is a perspective view of the garment of FIG. 8A, but with the left refastenable side seam open and the right refastenable side seam closed.

FIG. 8C is a perspective view of the garment of FIG. 8A, but with the right refastenable side seam open and the left refastenable side seam closed.

FIG. 8D is a perspective view of a garment of FIG. 8D, but with both refastenable side seams open, and the garment shown in a laid-open, relaxed configuration.

FIGS. 9A-9G are representative depictions of various stages of applying a refastenable garment in accordance with one embodiment of the present invention.

FIGS. 10A-10G are representative depictions of various stages of applying a refastenable garment in accordance with an alternative embodiment of the present invention.

FIGS. 11A-11F are a series of representative instructional graphic images as can be disposed on the package of FIG. 7.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION OF PARTICULAR EMBODIMENTS

The present invention relates to a method of applying a refastenable absorbent article to a subject; to a method for simplifying the application of a refastenable absorbent article to a subject; and to a package including secondary information for simplifying the application of a refastenable absorbent article to a subject. Reference to the Figures shall be made in describing various embodiments of the invention. It should be noted that the embodiments depicted in the Figures and described herein are merely representative examples of the method of the invention. The various embodiments of the invention are suitable for use in conjunction with disposable absorbent garments such as refastenable adult incontinence underwear, prefastened disposable diapers, refastenable disposable training pants or swim pants, refastenable disposable enuresis garments, and the like. For illustration purposes, various embodiments of the present method invention shall be described in conjunction with refastenable incontinence underwear.

Within the context of this specification, each term or phrase below will include the following meaning or meanings. Additional terms are defined elsewhere in the specification.

“Attached” refers to the joining, adhering, bonding, connecting, or the like, of two elements. Two elements will be considered to be attached together when they are attached directly to one another or indirectly to one another, such as when each is directly attached to intermediate elements.

“Disposable” refers to articles which are designed to be discarded after a limited use rather than being laundered or otherwise restored for reuse.

“Elastomeric” refers to a material or composite which can be elongated by at least 50% of its relaxed length and which will recover, upon release of the applied force, at least 20% of its elongation. It is generally preferred that the elastomeric material or composite be capable of being elongated by at least 100%, more preferably by at least 200%, of its relaxed length and recover, upon release of an applied force, at least 50% of its elongation.

“Longitudinal” and “transverse” have their customary meaning, as indicated by the longitudinal and transverse axes depicted in the Figures. The longitudinal axis lies in the plane of the article and is generally parallel to a vertical plane that bisects a standing wearer into left and right body halves when the article is worn. The transverse axis lies in the plane of the article generally perpendicular to the longitudinal axis.

These terms may be defined with additional language in the remaining portions of the specification.

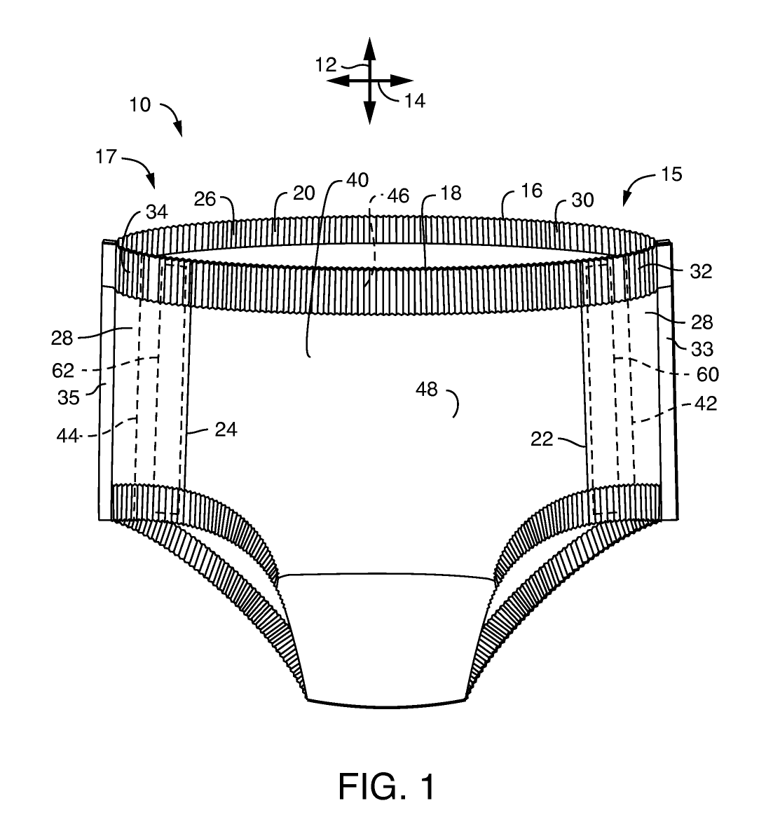

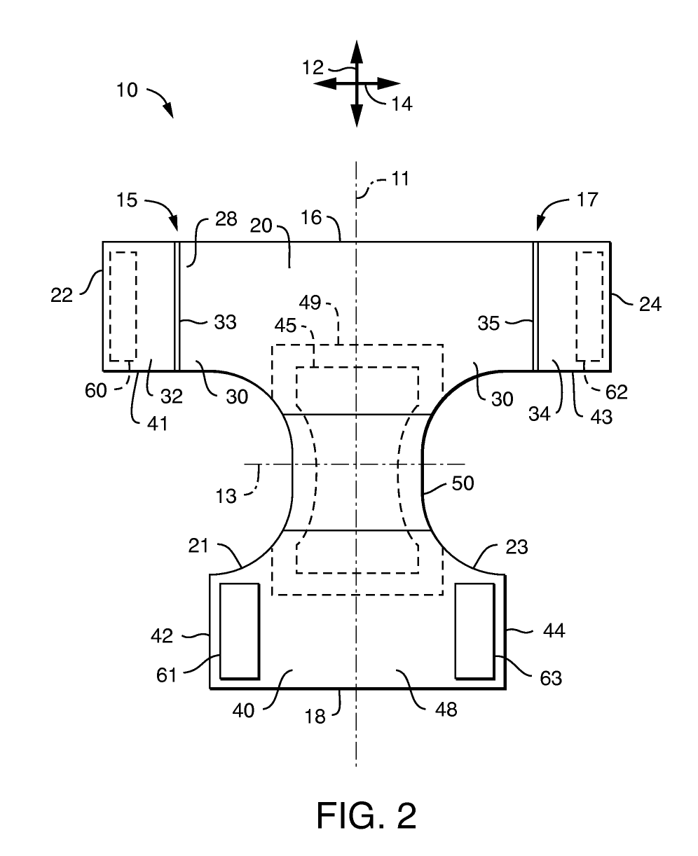

FIGS. 1-2 representatively illustrate a disposable absorbent garment suitable for use in conjunction with particular embodiments of the present invention. In particular embodiments, the garment 10 defines a longitudinal direction 12, a longitudinal axis 11, a transverse direction 14, and a transverse axis 13. The garment defines longitudinally spaced apart first and second waist edges 16, 18. Each waist edge 16, 18 extends generally in the transverse direction 14. In particular embodiments, the first waist edge 16 is a front waist edge, corresponding to the front of the wearer, and the back waist edge 18 is a back waist edge, corresponding to the back of the wearer. In other embodiments, the first and second waist edges 16, 18 instead correspond to back and front waist edges, respectively, as representatively illustrated in FIGS. 1 and 2.

The garment includes a first waist region 20 contiguous with the first waist edge 16. The first waist region 20 defines first and second side edges 22, 24 spaced apart from each other in the transverse direction 14. The first waist region 20 defines a first surface 26 and an opposing second surface 28. The first surface 26 can be a body-facing surface and the second surface 28 can be a clothing-facing surface, as representatively illustrated in FIGS. 1-2; alternatively, the first surface 26 can be a clothing-facing surface and the second surface 28 can be a body-facing surface. The garment further includes a second waist region 40 contiguous with the second waist edge 18. The second waist region 40 and defines first and second side edges 42, 44 spaced apart from each in the transverse direction 14. The second waist region defines a first surface 46 and an opposing second surface 48. The first surface 46 can be a body-facing surface and the second surface 48 can be a clothing-facing surface; alternatively, the first surface 46 can be a clothing-facing surface and the second surface 48 can be a body-facing surface.

In particular embodiments, the first waist region 20 is a front waist region, corresponding to the front of the wearer, and the second waist region 40 is a back waist edge, corresponding to the back of the wearer. In other embodiments, the first and second waist regions 20, 40 instead correspond to back and front waist regions, respectively, as representatively illustrated in FIGS. 1 and 2. The garment further includes a crotch region 50 that extends between and interconnects the first waist region 20 and the second waist region 40. The crotch region 50 can include an absorbent composite 49, such as an absorbent composite constructed of a fluid-absorbent core 45 of fluff and/or superabsorbent polymer sandwiched between a bodyside topsheet and a non-bodyside backsheet. Such absorbent composites are known in the art.

The article or garment 10 defines a first transverse side 15 and a second transverse side 17. The first transverse side 15 includes a first front side panel 21 and a first back side panel 41, and the second transverse side 17 includes a second front side panel 23 and a second back side panel 43. The first front side panel 21 is bordered by the first side edge 42, and the first back side panel 41 is bordered by the first side edge 22. Similarly, the second front side panel 23 is bordered by the second side edge 44, and the second back side panel 43 is bordered by the second side edge 24.

In particular embodiments, the first waist region 20 is at least partially elastomeric. In addition or in the alternative, the second waist region 40 is at least partially elastomeric. In particular embodiments, the first waist region 20 is substantially continuously transversely elastomeric from the first side edge 22 to the second side edge 24. Similarly, in particular embodiments, the second waist region 40 is substantially continuously transversely elastomeric from the first side edge 42 to the second side edge 44. In evaluating whether a waist region is substantially continuously transversely elastomeric, the region or regions to which any fastening component (described below) or absorbent composite is attached, or regions within which bonds (such as ultrasonic, heat, or pressure bonds) or seams are present, are ignored. In particular embodiments, the first waist region 20, the second waist region 40, or both, comprise an elastomeric nonwoven material, and preferably consist essentially of an elastomeric nonwoven material. In particular embodiments, the elastomeric nonwoven material comprises an elastomeric film sandwiched between two nonwoven facing layers. U.S. Pat. No. 7,803,244 issued Sep. 28, 2010 to Siqueira et al. and assigned to Kimberly-Clark Worldwide, Inc. discloses particular examples of elastomeric nonwoven composites suitable for use in conjunction with particular embodiments of the present invention. In other embodiments, the elastomeric nonwoven material can include elastomeric threads or ribbons sandwiched between two nonwoven layers.

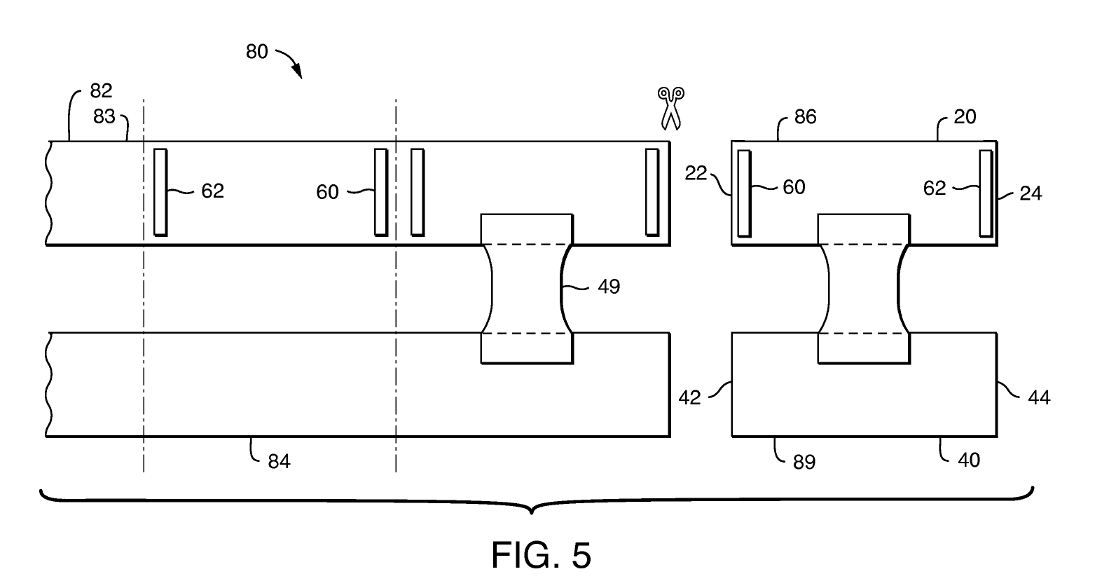

In particular embodiments, the first waist region 20 is formed by a unitary member that extends from the first side edge 22 of the first waist region 20 to the second side edge 24 of the first side region 20. Such an embodiment is depicted at the right side of FIG. 5. In other embodiments, the first waist region is formed by bonding together multiple pieces of material. For example, in the embodiment of FIGS. 1-2 and 4, the first waist region 20 (which is depicted as a back waist region) is comprised of a center section (such as a relatively wide center section 30), a first side section 32, and a second side section 34. The first side section 32 is attached to the center section 30 along a first side seam 33, and the second side section 34 is attached to the center section 30 along a second side seam 35. In such an embodiment, when the garment 10 is positioned on a wearer, the side seams 33, 35 align generally with the hips of the wearer. Note that in the embodiments of FIGS. 1-2, the first (back) waist region 20 of the garment 10, when configured in a pant-like configuration as shown in FIG. 1, would actually extend forward past the hip area to partially wrap around the front waist section of the wearer. In other words, the first and second side section 32, 34 are considered a part of the first (back) waist region, even though they are positioned on the front waist section of the wearer when the garment is donned.

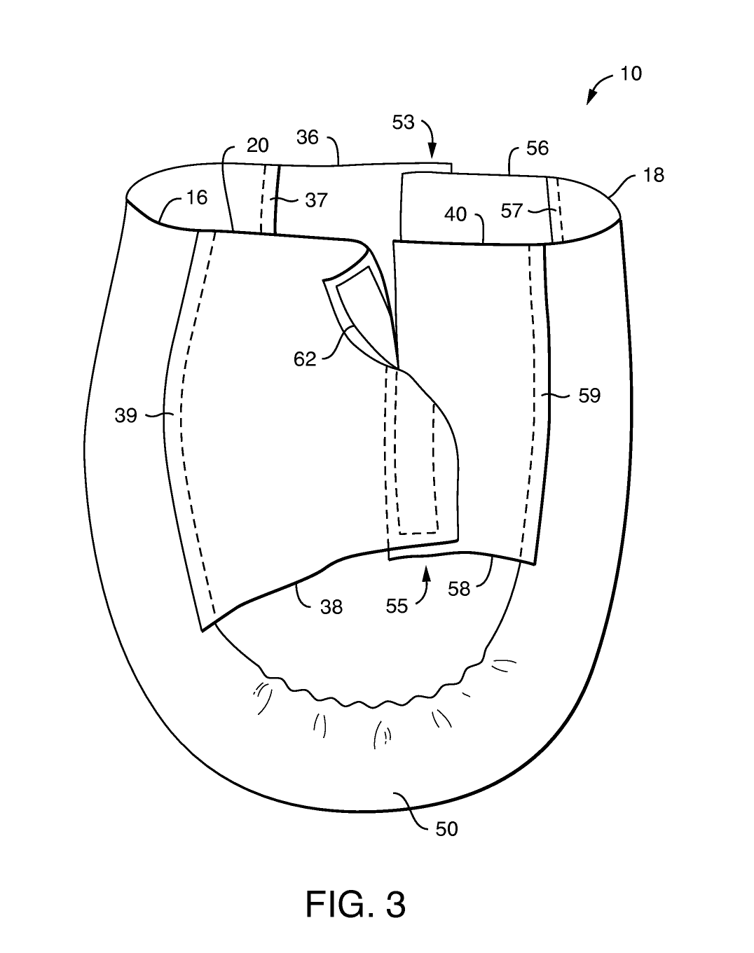

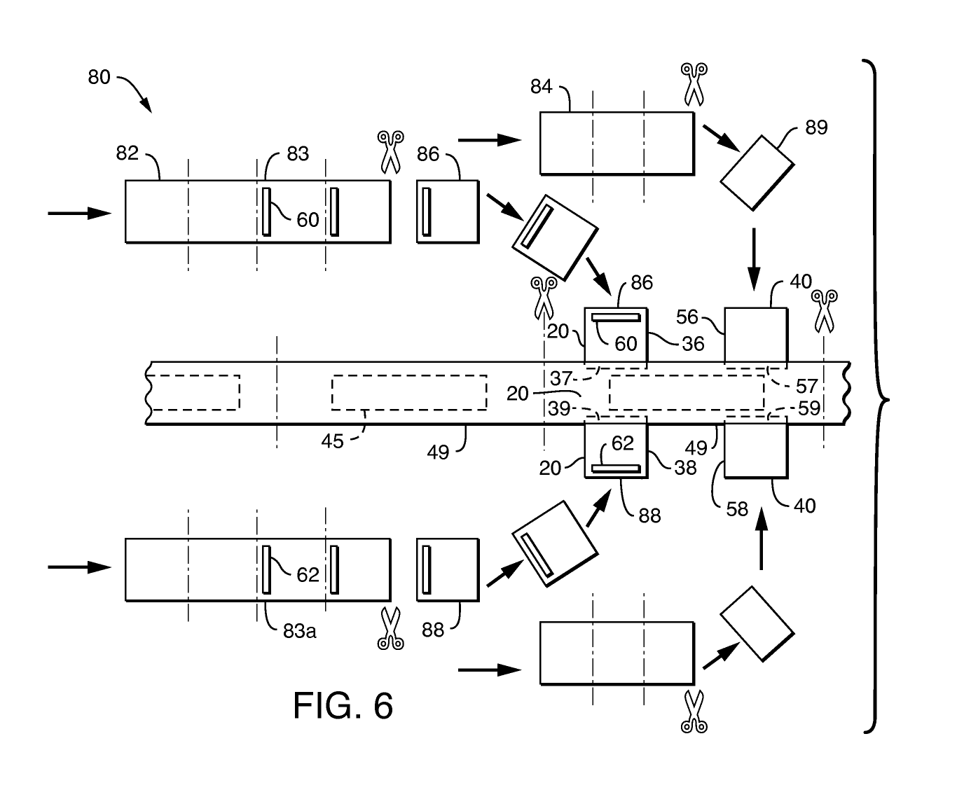

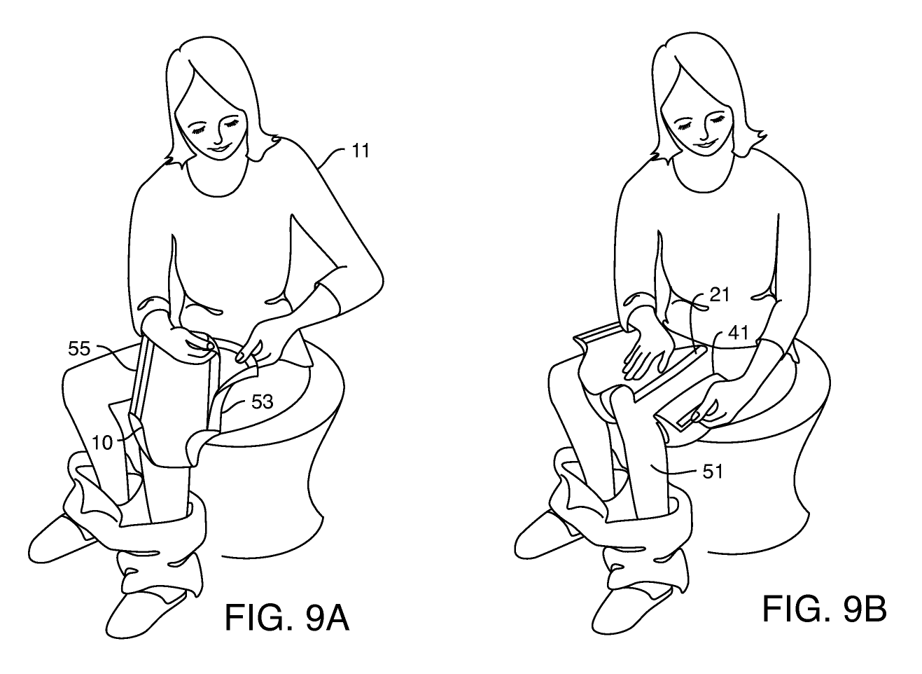

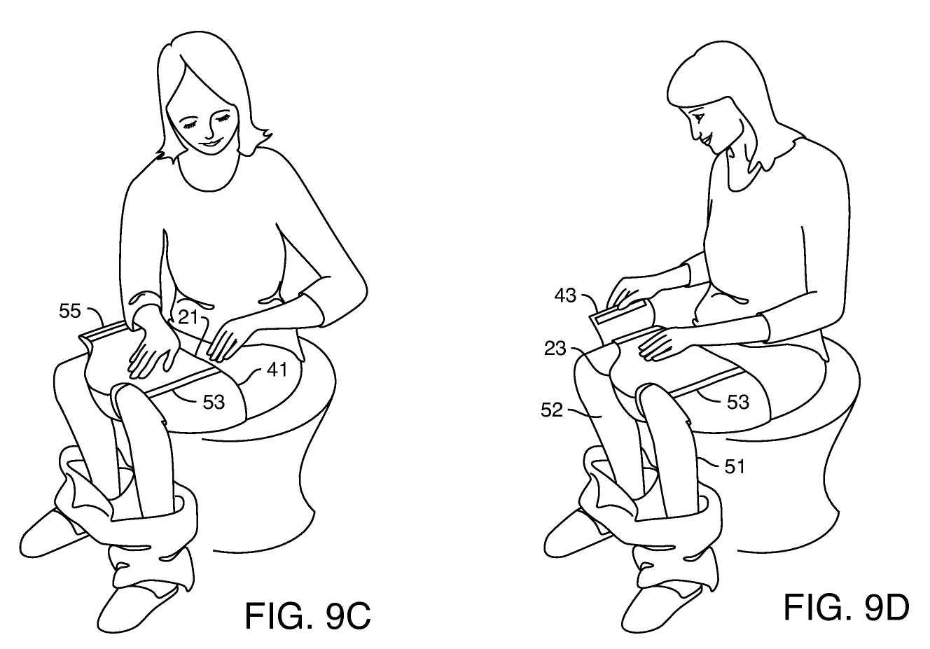

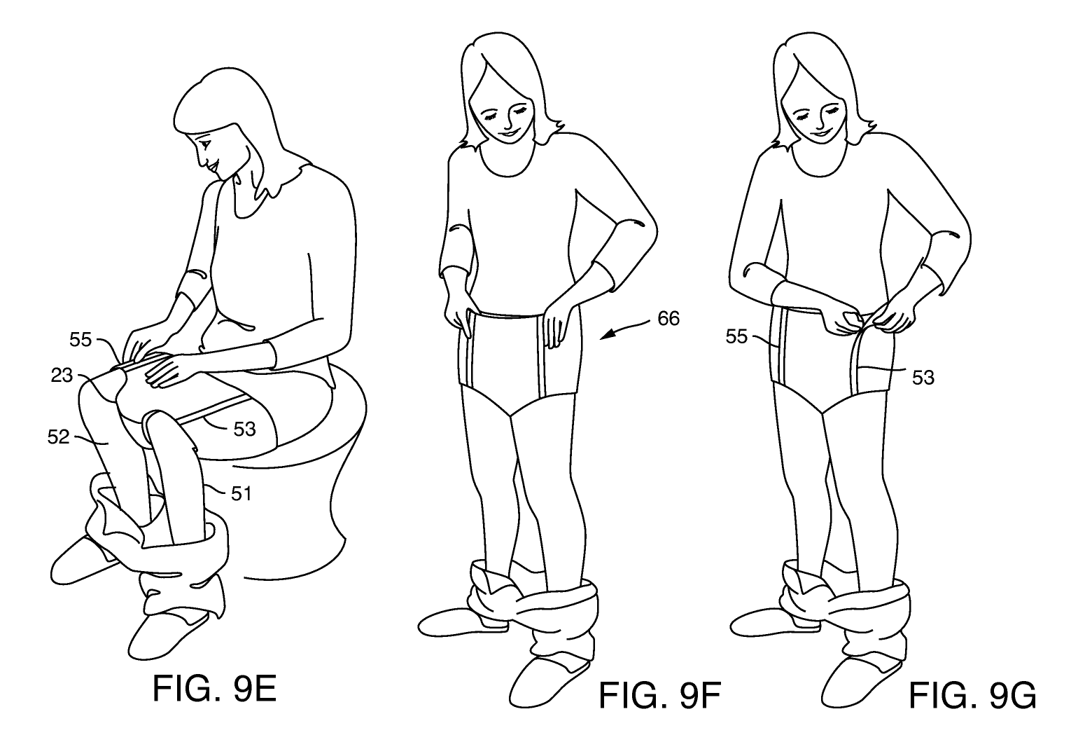

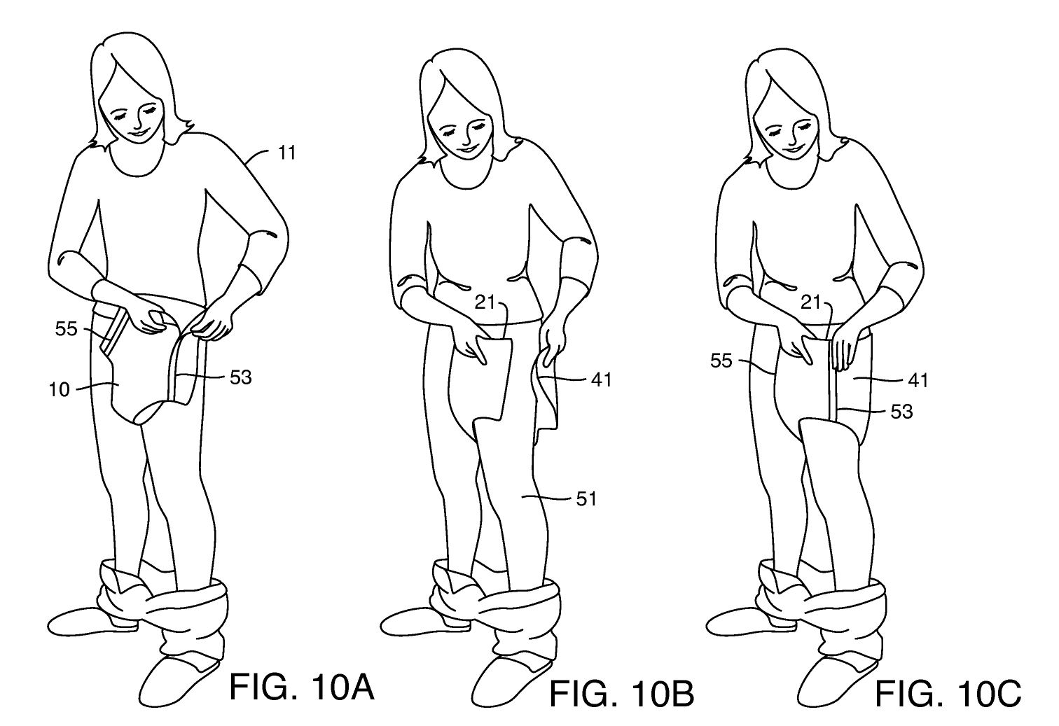

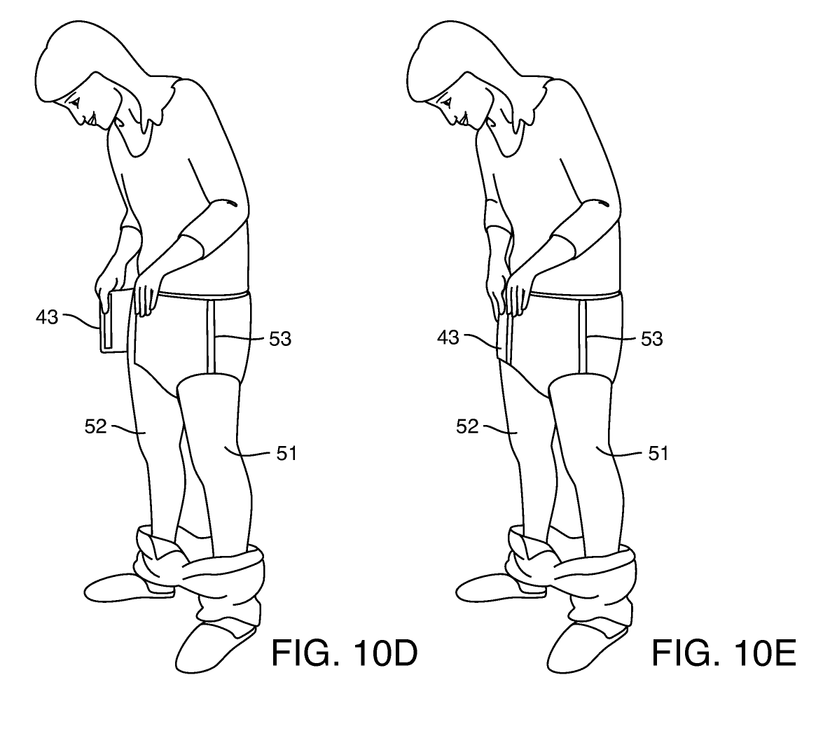

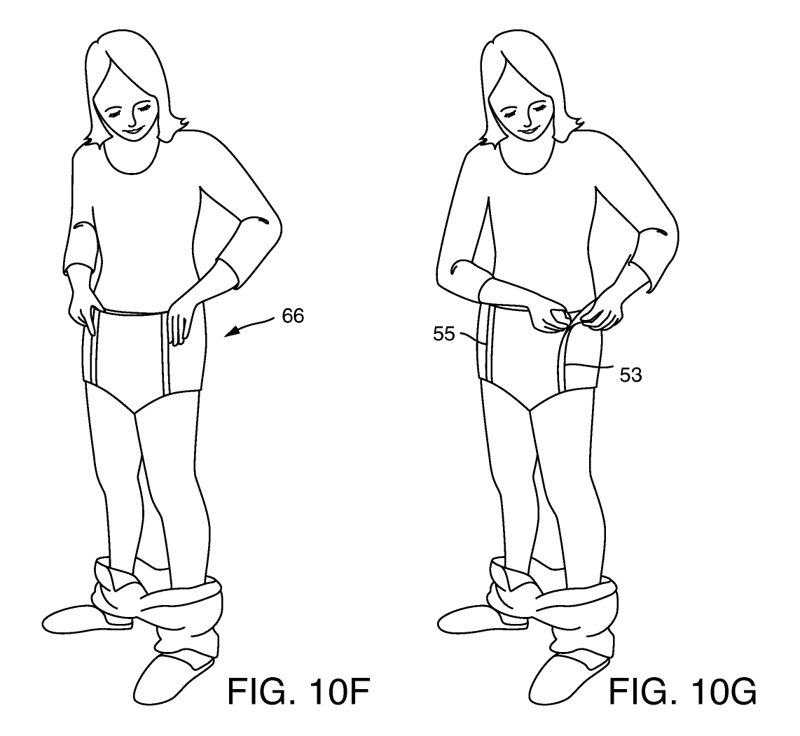

In an alternative embodiment, the first waist region 20 is formed by bonding opposing first and second side panels 36 and 38 to a central absorbent composite 49 along bond regions 37 and 39, as representatively illustrated in FIGS. 3 and 6. Similarly, the second waist panel is formed by bonding opposing third and fourth side panels 56 and 58 to the central absorbent composite 49 along bond regions 57 and 59. Such a garment may be formed into a pant-like configuration by joining the side panels 36 and 38 to the side panels 56 and 58 along a pair of refastenable side seams 53, 55, as representatively illustrated in FIG. 3. In some embodiments, the refastenable side seams 53, 55 are positioned such that they would align with the wearer's hip in use (e.g., FIG. 3); in other embodiments, the refastenable side seams 53, 55, are positioned such that they would align with the front of wearers hips, in the side regions of the abdomen (e.g., FIG. 1, and FIGS. 9F and 10F). In particular embodiments, each side panel, 36/38/56/58 comprises an elastomeric nonwoven material, and preferably consists essentially of an elastomeric nonwoven material. In particular embodiments, the central absorbent composite 49 comprises an absorbent core 45 sandwiched between a body-side topsheet layer and a non-bodyside backsheet layer (FIG. 6).

The garment 10 further comprises a first fastening component 60 attached to the first surface 26 of the first waist region 20 adjacent its first side edge 22, and further comprises a second fastening component 62 attached to the first surface 26 of the first waist region 20 adjacent its second side edge 24. Referring to FIG. 2, the garment can optionally include first and second mating fastening components 61, 63. Fastening components 60, 62 are adapted to releasably and refastenably engage the opposite waist region, such as by engaging the elastomeric nonwoven side panel material, or by engaging mating fastening components 61, 63, respectively. Example of suitable fastening components include but are not limited to mechanical fasteners, such as hook or mushroom type fastening components. Examples of suitable mating fastening components include nonwoven fiber matrix or loop type fastening components.

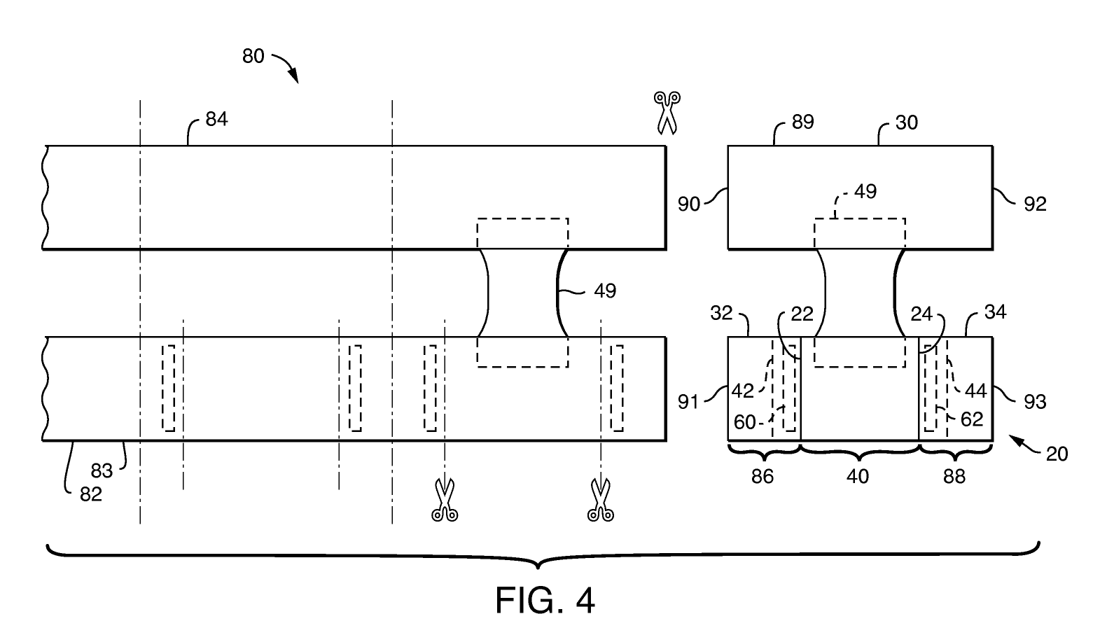

FIGS. 4-6 representatively illustrate processes suitable for constructing disposable absorbent garments suitable for use in conjunction with particular embodiments of the present invention, such as the garments described above. In particular embodiments, the process 80 includes providing at least a first elastomeric waist panel web 82. The elastomeric waist panel web can comprise an elastomeric nonwoven composite, and elastomeric film/nonwoven laminate, an elastomeric filament/nonwoven, or the like. The method 80 can further include stretching the elastomeric waist panel web to create a stretched waist panel web 83. The method 80 further comprises attaching a first fastening component 60 to the stretched waist panel web 83. The method further includes severing (such as by cutting) the first waist panel web 82 adjacent the first fastening component 60 to create a first waist region first portion 86. The method further includes incorporating the first waist region first portion 86 into a first waist region 20 of the garment 10.

The first waist region first portion 86 can comprise the entirety of the first waist region 20, or can comprise only a part of the first waist region 20. For example, in the embodiment representatively illustrated in FIG. 5, the first waist region first portion 86 comprises the entirety of the first waist region 20, such that the first waist region 20 is defined by a unitary member that extends from the first side edge 22 of the first waist region 20 to the second side edge 24 of the first side region 20. Alternatively, in the embodiments of FIGS. 4 and 6, the first waist region first portion 86 comprises only one side part of the first waist region 20.

Still referring to FIGS. 4 and 5, in particular embodiments the process further comprises attaching a non-extensible second fastening component 62 to a stretched waist panel web, such as to the same stretched waist panel web 83 (FIGS. 4 and 5), or to an additional waist panel web 83a (FIG. 6). The process in this embodiment further includes severing the waist panel web 83/83a adjacent the second fastening component to create a first waist region second portion 88, and incorporating the first waist region second portion 88 into the first waist region 20 of the garment 10.

In particular embodiments, the process 80 further comprises providing a second waist panel web, such as a second elastomeric waist panel web 84. The second elastomeric waist panel web 84 can comprise an elastomeric nonwoven composite, and elastomeric film/nonwoven laminate, an elastomeric filament/nonwoven, or the like. The method 80 can further include stretching the second elastomeric waist panel web. The method 80 can further comprise attaching a mating fastening component or components (not shown) to the second waist panel web 84. The method further includes severing (such as by cutting) the second waist panel web 84 to create a second waist region portion 89. The method further includes incorporating the second waist region portion 89 into a second waist region 40 of the garment 10.

In the process embodiment of FIG. 4, edges 90, 92 may be permanently bonded to edges, 91, 93, respectively, to form the pant-like configuration representatively illustrated in FIGS. 1-2 as a garment 10 having side seams 33, 35. In the process embodiment of FIG. 5, the fastening components 60, 62 may be refastenably engaged with the second waist region 40 in close proximity to the first and second side edges 42, 44, respectively, to form a garment having a pant-like configuration. In the process embodiment of FIG. 6, the fastening component 60 can be refastenably engaged to the third side panel 56 and the fastening component 62 can be refastenably engaged to the fourth side panel 58 to form a garment having the pant-like configuration representatively illustrated in FIG. 3 as a pant-like garment 10.

In particular embodiments, the garments 10 are constructed and pre-assembled in their pull-on style, wear configuration, with the article fastening system releasably attaching the first and second waist regions 20, 40. In particular embodiments, the garment 10 may be provided in a closed, pant-like configuration, and a user would be required to separate the first waist region 20 from the second waist region 40 by tearing along one or more lines of perforations. Examples of such articles are disclosed in U.S. Pat. No. 6,838,040 to Mlinar et al., which is incorporated by reference in its entirety to the extent not inconsistent herewith. In other embodiments, the garments are constructed and pre-assembled in a non-pull-on, non-prefastened configuration, akin to a traditional diaper. In still other embodiments, the garments are constructed and pre-assembled with the first transverse side 15 configured in a prefastened, closed configuration, and with the second transverse side 17 configured in a non-prefastened, open configuration.

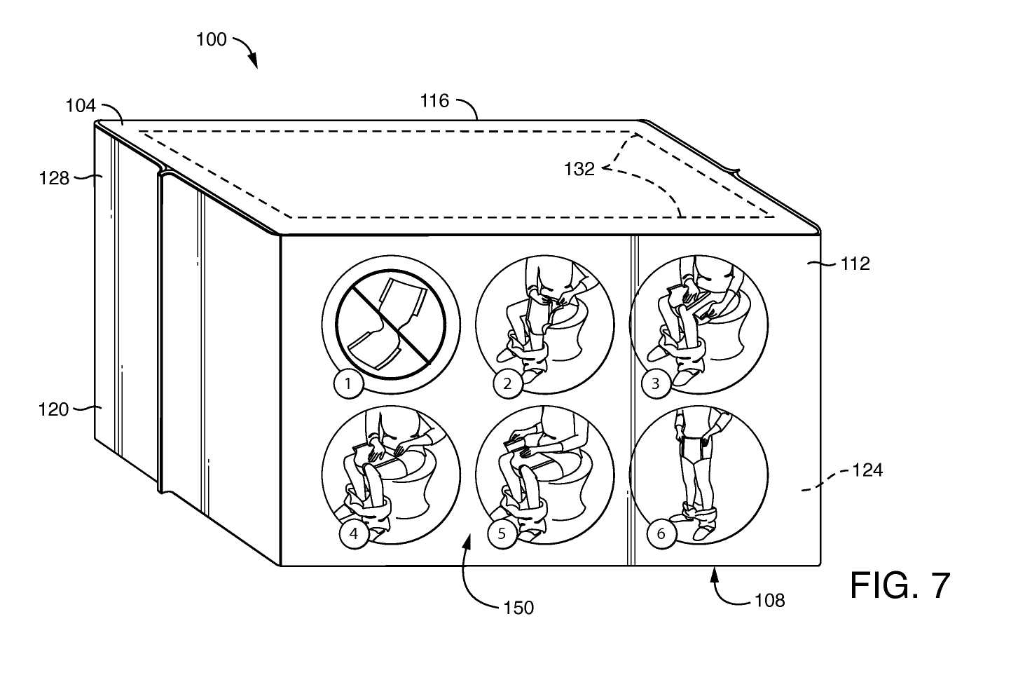

In particular embodiments, the present invention is directed to a package 100 of prefastened absorbent articles 10, representatively illustrated in FIG. 7. The package 100 includes a plurality of prefastened absorbent articles 10 disposed within a sealed bag, carton, or other container. The package 100 defines an interior space to receive the absorbent articles 10. The package 100 can but need not include a window for viewing the absorbent articles 10. In particular embodiments, the flexible package 100 has a top surface 104, a bottom surface 108, a front wall 112, a back wall 116, and a first side wall 120 and a second side wall 124. The first side wall 120 and the second side wall 124 are opposed to each other; as are the front wall 112 and the back wall 116, and the top surface 104 and the bottom surface 108. As shown, the front wall 112, the back wall 116, the two side walls 120, 124, the top surface 104, and the bottom surface 108 are directly or indirectly connected together to form the package 100. The package 100 includes an exterior surface 128. The package 100 can optionally include one or more lines of weakness 132 that are designed to be broken open by the consumer to access the garments housed in the interior space.

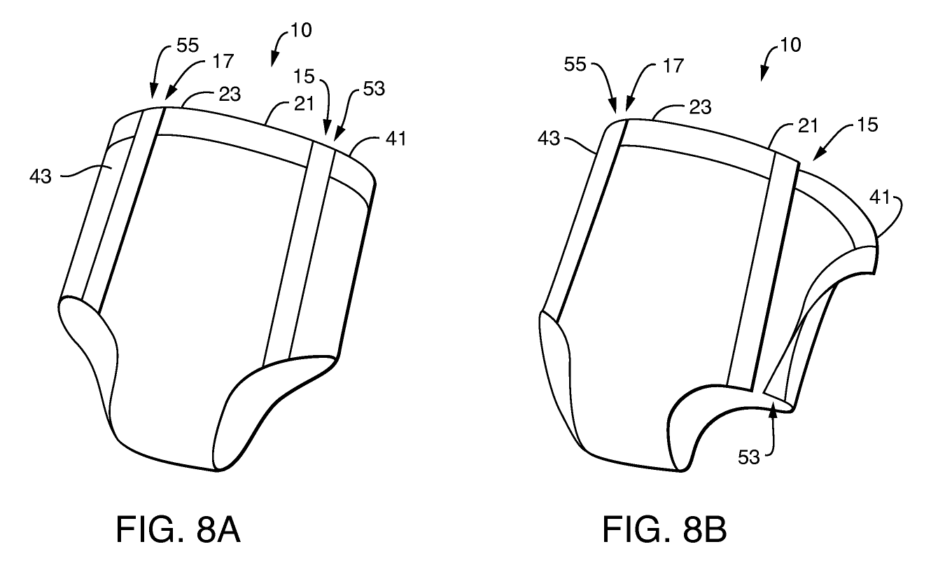

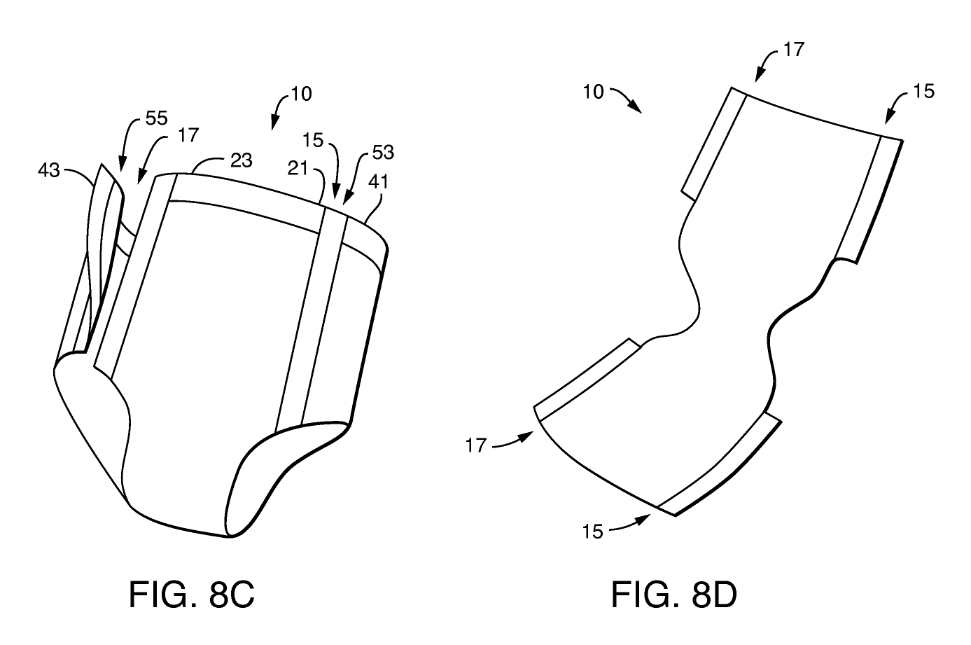

Referring to FIGS. 8-10, the invention in one aspect pertains to a method for increasing the donning efficiency of a pant-like absorbent garment 10 having refastenable seams 53, 55 for ease of removal and donning of the absorbent garment 10. Existing methods for donning an absorbent garment 10 suffer from various deficiencies. One conventional approach is to remove one's shoes and bottom garment, and to thereafter draw up the pant-like garment 10 similarly to regular underwear. This method suffers from the inconvenience of having to remove shoes and bottom garments to apply the article. A second conventional approach is to configure the garment with both refastenable side seams in an open configuration, and to thereafter apply the product like a traditional diaper—i.e., by sliding the garment 10 under the supine individual. However, it is difficult to employ this method when one is applying the garment to oneself. A third approach is to unfasten both sides of the prefastened garment, to thread the opened garment between the legs of the subject, and to then refasten both sides of the product around the legs of the wearer. This method is also not ideal for certain users; when both sides of the article are unfastened, it can be difficult and confusing to fasten the article around a leg without accidentally fastening the front panel of one side of the opened article to the back panel of the opposite side of the opened article. Further, the exposed “hook or mushroom” style fasteners on one side of the opened garment can inadvertently engage the wearer's clothing or the disposable article itself while the user is working to fasten the other side about his or her leg.

The present inventors, through their research, have established that, in particular embodiments, a more efficient donning process is to fasten one side of the article 10 around one leg while the other side of the article is configured in a fastened, closed condition, and to thereafter open the remaining side of the article and to fasten such remaining side around the remaining leg.

Accordingly, the present invention in one aspect relates to a method for applying a refastenable absorbent article 10 to a subject 11. The method comprises configuring the article such that the first transverse side 15 is open and such that the second transverse side 17 is closed, as is representatively illustrated in FIG. 8B. Prior to this first configuring stage, the article may be in a fully closed configuration, as shown in FIG. 8A. “Closed” as used herein means that both (or, more generically, all) refastenable side seams are engaged, such that the pant first exists in a pant-like, pull-on form when removed from the package 100. Alternatively, prior to the first configuring stage, the article may be in a fully open configuration, as shown in FIG. 8D. “Open” means that one or both (or, more generically, all) refastenable side seams are disengaged, such that the pant first exists in a diaper-like, non-pull-on form when removed from the package 100. In a preferable embodiment, the aforementioned first configuring step includes beginning with a garment form in which both the first and second transverse sides are closed (FIG. 8A), and thereafter opening the first transverse side (FIG. 8B). For example, FIG. 9A depicts a sitting subject 11, starting with a closed article (FIG. 8A), opening the prefastened, refastenable side seam 53, and leaving the opposite side seam 55 in its initial, prefastened condition. FIG. 10A shows the same step as FIG. 9A, except that the subject is shown in the standing position.

Referring to FIGS. 9B and 10B, the first front and back side panels 21, 41 are next positioned around the first leg 51 (such as around the thigh portion of the first leg 51) such that the first front side panel 21 meets the first back side panel 41. During this step, the opposite side seam 55 remains closed. Referring to FIGS. 9C and 10C, the first front side panel 21 is thereafter fastened to the first back side panel 41 to at least partially, and preferably completely, refasten the refastenable side seam 53. In certain embodiments, it is advantageous to use the subject's thigh as an “anvil” against which to press the refastenable side seam 53 as it is refastened. At this stage in the method, both sides 53, 55 are in a closed condition (FIG. 8A), although only the first leg 51 extends through the article 10. Again, FIGS. 10B and 10C show the same steps as FIGS. 9B and 9C, except that the subject is shown in the standing position.

Referring to FIGS. 9D and 10D, the method includes thereafter opening the second transverse side 17, such that the article 10 takes on the configuration representatively depicted in FIG. 8C. For example, FIG. 9D depicts a sitting subject 11, opening the prefastened, refastenable side seam 55 (see FIG. 9C), but leaving the opposite side seam 53 in its fastened condition (the subject having just fastened side seam 53 about the first leg 51). FIG. 10D shows the same step as FIG. 9D, except that the subject is shown in the standing position. As further representatively illustrated in FIGS. 9D and 10D, the method includes thereafter positioning the second front and back side panels 23, 43 around the second leg 52 (such as around the thigh portion of the second leg 52) such that the second front side panel 23 meets the second back side panel 43. Referring to FIGS. 9E and 10E, the method in particular embodiments includes thereafter at least partially, and preferably completely, fastening the second front side panel 23 to the second back side panel 43 to thereby refasten the refastenable side seam 55. In certain embodiments, it is advantageous to use the subject's thigh as an “anvil” against which to press refastenable side seam 55 as it is refastened. At this stage in the method, both sides 53, 55 are again in a closed condition (FIG. 8A), and both the first leg 51 and the second leg 52 now extend through the article 10.

Referring to FIGS. 9F and 10F, the method includes thereafter drawing the article 10 up into a wear position 66 on the subject 11. Typically, but not necessarily, the subject would stand during this step. Optionally, the subject or caregiver may thereafter make adjustments to the refastenable side seam 53 and/or the refastenable side seam 55, such optional step being representatively illustrated in FIGS. 9G and 10G.

Note that the preceding sequence of steps could also be performed in conjunction with a subject in the supine position (as opposed to a sitting or standing position), such as by a caregiver performing the various steps on a bed-ridden subject.

One difficulty in promoting the widespread use of the application techniques described above is the challenge of teaching the method to a subject or caregiver. Since packages 100 of diapers, training pants, and incontinence garments 10 typically do not include instructions, users apply such products in a way that is familiar to them, such as via any of the three deficient approaches described earlier.

Accordingly, in another aspect, the present invention is directed to a method for simplifying the application of a refastenable absorbent article to a subject. In particular embodiments, the method includes manufacturing a plurality of refastenable absorbent articles. Examples of techniques suitable for manufacturing refastenable absorbent articles, such as prefastened, refastenable absorbent articles, are disclosed in U.S. Pat. No. 6,838,040 to Mlinar et al., which is incorporated by reference in its entirety to the extent not inconsistent herewith. The method for simplifying application further includes assembling the plurality of articles 10 into a package 100. The method for simplifying application further includes instructing a consumer to follow a series of application steps, such as those described above in conjunction with FIGS. 8-10.

Retraining a population of refastenable incontinence garment users may require more than text instructions, which may go unnoticed. The present inventors have learned that prominently-displayed secondary information such as graphical images illustrating the techniques of the method can in particular embodiments be more effective than text instructions in teaching the method.

For effective retraining, instructions illustrating particular embodiments of the method of the present invention can be presented in the form of one or more graphic images 150. The graphic images 150 can be presented to the user on a package 100, or on an insert disposed within or on the package 100. In one aspect of the present disclosure, the method is illustrated in a series of graphic images, such as those of FIGS. 9 and 10, each highlighting a particular step in the method.

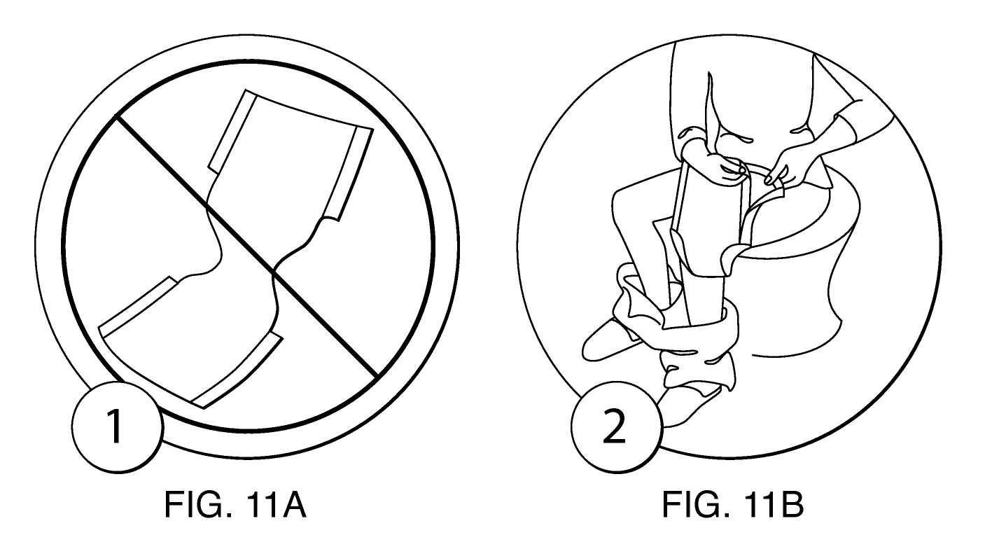

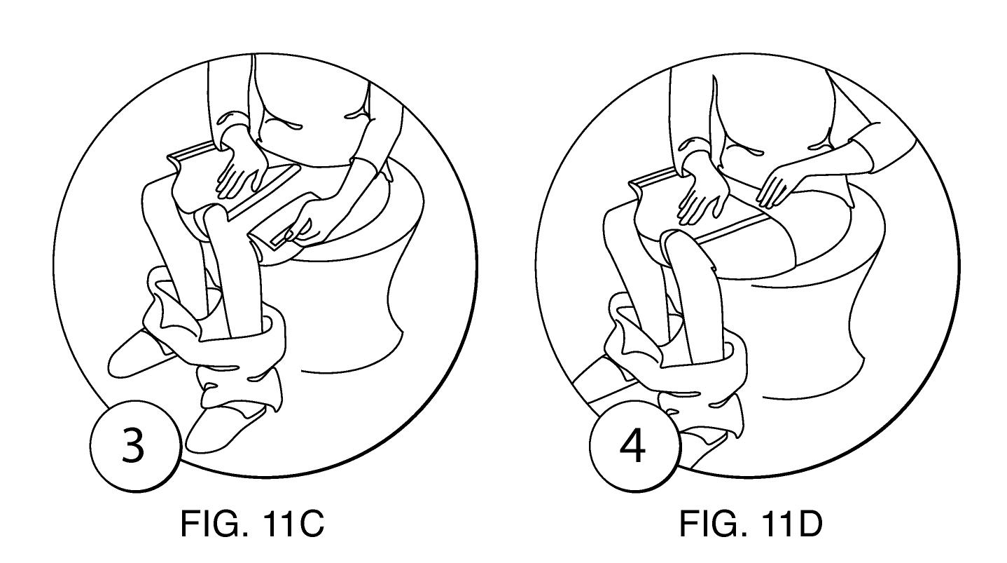

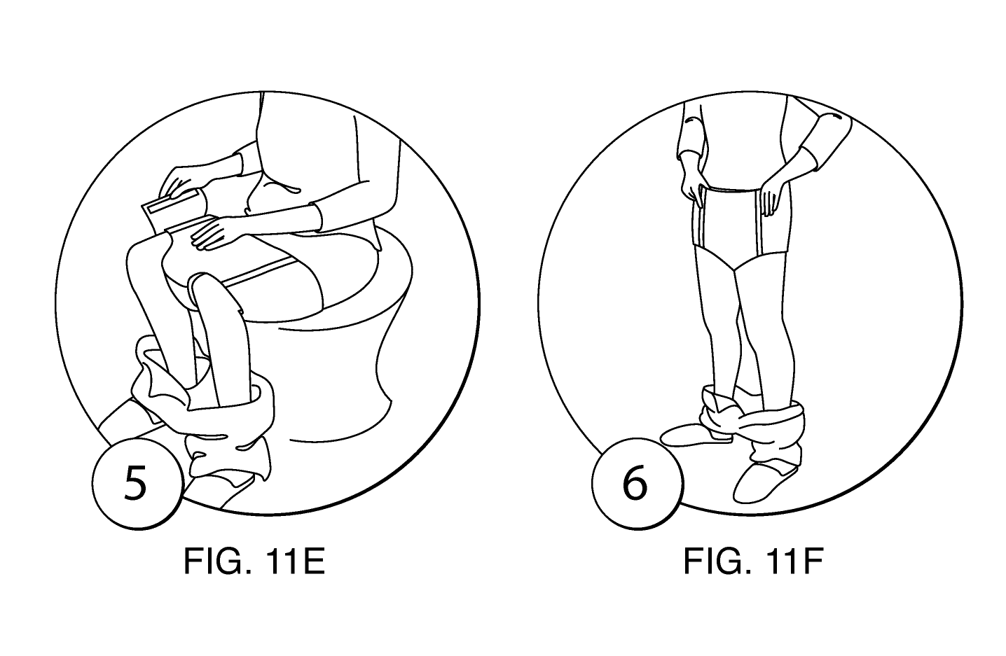

In particular embodiments of the method for simplifying application by instructing the consumer, such instructing comprises providing graphic images 150 on the package 100, as representatively illustrated in FIG. 7. The graphic images 150 illustrate the steps, or a variant of the steps, set forth above. One example of a series of graphic images 150 setting forth various suitable steps appears in FIGS. 7 and 11. In particular embodiments, the method comprises graphic images that illustrate (1) opening the first transverse side 15 of the article 10 while leaving the second transverse side 17 of the article closed; (2) thereafter positioning the first front and back side panels 21, 41 around the first leg 51 such that the first front side panel 21 meets the first back side panel 41; (3) thereafter fastening the first front side panel 21 to the first back side panel 41; (4) thereafter opening the second transverse side 17; (5) thereafter positioning the second front and back side panels 23, 43 around the second leg 52 such that the second front side panel 23 meets the second back side panel 43; and thereafter fastening the second front side panel 23 to the second back side panel 43.

In particular embodiments, the method includes providing a graphic image that illustrates positioning the first front and back side panels around a first thigh and positioning the second front and back side panels around a second thigh. In particular embodiments, the method includes providing a graphic image that illustrates drawing the article from around the first and second thighs up into a wear position 66 on the subject 11. In particular embodiments, at least one of the graphic images includes a depiction of the subject 11 without bottom garments removed.

In an alternative embodiment, the consumer is instructed in the application steps via the aid of a video-based demonstration. For example, a video-based demonstration of the application techniques can be supplied via an internet website, via a CD-ROM, via a flash memory stick, or the like. Alternatively, the consumer may be instructed via an advertisement.

In one representative example of the package aspect of the present invention, shown in FIG. 7 and FIGS. 11A-11F, a series of graphic images on the package 100 illustrate various steps in the method of particular embodiments of the present invention. A first graphic image, shown in FIG. 11A, indicates to the subject that the prefastened garment that he or she has just removed from the package should not be entirely unfastened to an open configuration, as indicated by the prohibition sign superposed over an open garment. A second graphic image, shown in FIG. 11B, shows the subject opening the refastenable seam 53 on the first transverse side 15. A third graphic image, shown in FIG. 11C, shows the subject wrapping the first front and back side panels 12, 23 around the first leg 51. A fourth graphic image, shown in FIG. 11D, shows the subject closing the refastenable side seam 53. A fifth graphic image, shown in FIG. 11E, indicates to the subject to repeat the previous four steps on the second transverse side 17. A sixth graphic, shown in FIG. 11F, shows the subject standing and drawing up the fully fastened article. Other combinations of graphic images are possible, and can include only a portion of the steps just described, or a different combination of steps, or steps not included in the graphic images just described. The graphic images set forth in FIGS. 7 and 11 demonstrate that the method can be performed without removing the subject's bottom garment(s) or shoes.

In one embodiment, the package 100 includes an exterior surface 128, and a plurality of refastenable absorbent articles disposed within the package 100. The package includes a first graphic image associated with the package illustrating positioning the first front and back side panels 21, 23 around the first leg 51 such that the first front side panel 21 meets the first back side panel 23, wherein the second transverse side 17 is depicted as closed. The package further includes a second graphic image associated with the package illustrating opening the second transverse side 17. The package further includes a third graphic image associated with the package illustrating positioning the second front and back side panels 41, 43 around the second leg 52 such that the second front side panel 41 meets the second back side panel 43. Examples of the first, second, and third graphic images described in this paragraph include FIGS. 9B, 9D, and 9E, respectively. Desirably, the first graphic image is shown sequentially before the second graphic image, and the second graphic image is shown sequentially before the third graphic image. In one embodiment, each of the graphic images is disposed on the exterior surface 128 of the package 100. Desirably, each refastenable absorbent article 10 of the plurality of refastenable absorbent articles is disposed within the package 100 in a closed configuration.

It will be appreciated that details of the foregoing embodiments, given for purposes of illustration, are not to be construed as limiting the scope of this invention. Although only a few exemplary embodiments of this invention have been described in detail, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

We claim:

1. A method for applying a refastenable absorbent article to a subject, the article having first and second transverse sides, the first transverse side having first front and back side panels and the second transverse side having second front and back side panels, the subject having first and second legs, first and second thighs, the method comprising:
configuring the article such that the first transverse side is open and such that the second transverse side is closed;
thereafter positioning the first front and back side panels around the first leg such that the first front side panel meets the first back side panel, and thereafter fastening the first front side panel to the first back side panel;
thereafter opening the second transverse side;
thereafter positioning the second front and back side panels around the second leg such that the second front side panel meets the second back side panel, and thereafter fastening the second front side panel to the second back side panel; and
thereafter drawing the article up into a wear position on the subject.

2. The method of claim 1, wherein said positioning around the first and second legs comprises positioning around the first and second thighs, respectively.

3. The method of claim 2, wherein said positioning around the first and second thighs occurs while the subject is sitting.

4. The method of claim 1, wherein said configuring comprises beginning with a configuration in which both the first and second transverse sides are closed, and thereafter opening the first transverse side.

5. The method of claim 4, where said article comprises a first waist region and second waist region, and wherein said configuring comprises separating the first waist region from the second waist region by tearing along one or more lines of perforations.

6. A method for simplifying the application of a refastenable absorbent article to a subject, the article having first and second transverse sides, the first transverse side having first front and back side panels and the second transverse side having second front and back side panels, the subject having first and second legs, first and second thighs, the method comprising:
manufacturing a plurality of refastenable absorbent articles;
assembling the plurality of articles into a package; and
instructing a consumer to
configure the article such that the first transverse side is open and such that the second transverse side is closed;
position the first front and back side panels around the first leg such that the first front side panel meets the first back side panel, and thereafter fasten the first front side panel to the first back side panel;
thereafter open the second transverse side; and
thereafter position the second front and back side panels around the second leg such that the second front side panel meets the second back side panel, and thereafter fasten the second front side panel to the second back side panel.

7. The method of claim 6, wherein the plurality of refastenable absorbent articles are assembled into said package in a closed configuration.

8. The method of claim 6, wherein the plurality of refastenable absorbent articles are assembled into said package in an open configuration.

9. The method of claim 6, wherein the instructing comprises providing graphic images.

10. The method of claim 9, further comprising disposing the graphic images on the package.

11. The method of claim 9, further comprising providing graphic images that illustrate
opening the first transverse side of the article while leaving the second transverse side of the article closed;
thereafter positioning the first front and back side panels around the first leg such that the first front side panel meets the first back side panel;
thereafter fastening the first front side panel to the first back side panel;
thereafter opening the second transverse side;
thereafter positioning the second front and back side panels around the second leg such that the second front side panel meets the second back side panel; and
thereafter fastening the second front side panel to the second back side panel.

12. The method of claim 11, further comprising providing a graphic image that illustrates positioning the first front and back side panels around the first thigh and positioning the second front and back side panels around the second thigh.

13. The method of claim 6, further comprising instructing the consumer to tear along a line of weakness so as to configure the article such that the first transverse side is open and such that the second transverse side is closed.

14. The method of claim 6, wherein the instructing comprises supplying video-based instructions selected from the group of an internet website, a CD-ROM, or a flash memory stick.

15. A method for simplifying the application of a refastenable absorbent article to a subject, the article having first and second transverse sides, the first transverse side having first front and back side panels and the second transverse side having second front and back side panels, the subject having first and second legs, first and second thighs, the method comprising:
manufacturing a plurality of refastenable absorbent articles;
assembling the plurality of articles into a package;
providing a first graphic image associated with the package illustrating positioning the first front and back side panels around the first leg such that the first front side panel meets the first back side panel, wherein the second transverse side is depicted as closed;
providing a second graphic image associated with the package illustrating opening the second transverse side; and
providing a third graphic image associated with the package illustrating positioning the second front and back side panels around the second leg such that the second front side panel meets the second back side panel,
wherein the first graphic image is shown sequentially before the second graphic image, and wherein the second graphic image is shown sequentially before the third graphic image.

16. The method of claim 15, further comprising providing a fourth graphic image associated with the package illustrating drawing the article up into a wear position on the subject, wherein at least one of the graphic images includes a depiction of the subject without bottom garments removed.

17. The method of claim 15, wherein the first, second, and third graphic images are disposed on the outer surface of the package.

18. A package including secondary information for simplifying the application of a refastenable absorbent article to a subject, the article having first and second transverse sides, the first transverse side having first front and back side panels and the second transverse side having second front and back side panels, the subject having first and second legs, first and second thighs, the package comprising:
an outer surface;
a plurality of refastenable absorbent articles disposed within the package;
a first graphic image associated with the package illustrating positioning the first front and back side panels around the first leg such that the first front side panel meets the first back side panel, wherein the second transverse side is depicted as closed;
a second graphic image associated with the package illustrating opening the second transverse side; and
a third graphic image associated with the package illustrating positioning the second front and back side panels around the second leg such that the second front side panel meets the second back side panel,
wherein the first graphic image is shown sequentially before the second graphic image, and wherein the second graphic image is shown sequentially before the third graphic image.

19. The package of claim 18, wherein the first, second, and third graphic images are disposed on the outer surface of the package.

20. The package of claim 18, wherein each refastenable absorbent article of the plurality of refastenable absorbent articles is disposed within the package in a closed configuration.
